(19) United States
(12) Patent Application Publication
(10) Pub. No.: US 2018/0016226 A1
(43) Pub. Date: Jan. 18, 2018

(54) 4-AZIDOBUTYLAMINES AND PROCESSES FOR PREPARING

(71) Applicant: CEMPRA PHARMACEUTICALS, INC., CHAPEL HILL, NC (US)

(72) Inventors: Stephen E. SCHNEIDER, Raleigh, NC (US); David Eugene PEREIRA, Apex, NC (US)

(21) Appl. No.: 15/548,134

(22) PCT Filed: Feb. 5, 2016
(86) PCT No.: PCT/US16/16707

§ 371 Date: Aug. 2, 2017

Related U.S. Application Data
(60) Provisional application No. 62/112,672, filed on Feb. 6, 2015.

(51) Int. Cl. C07C 247/04; C07C 309/04; C07C 63/08; C07C 59/265; C07C 59/255; C07C 57/15; C07C 55/08; C07C 55/07; C07C 53/10; C07C 309/30; C07C 53/06

(52) U.S. Cl. CPC C07C 247/04; C07C 309/30; C07C 309/04; C07C 63/08; C07C 59/265; C07C 53/06; C07C 57/15; C07C 55/08; C07C 55/07; C07C 53/10; C07C 59/255

(57) ABSTRACT

Neat 4-azidobutylamine and sails of 4-azidobutylamine and processes for producing the same are described herein. Amines represent a large class of organic compounds containing a basic nitrogen atom having a lone pair of electrons and one or more substituent groups. Many amines are used as precursors and feedstocks in a wide variety of industries such as textiles, agriculture, plastics, and pharmaceuticals. One such amine is 4-azidobutylamine, N3-(CH2)4NH2, an amine of butane that also includes an azide.

### BACKGROUND AND SUMMARY OF THE INVENTION

Amines represent a large class of organic compounds containing a basic nitrogen atom having a lone pair of electrons and one or more substituent groups. Many amines are used as precursors and feedstocks in a wide variety of industries such as textiles, agriculture, plastics, and pharmaceuticals. One such amine is 4-azidobutylamine, N₃—(CH₂)₄—NH₂, an amine of butane that also includes an azide.

It has been reported that current processes for producing 4-azidobutylamine result in poor quality product and/or product having a limited shelf life. In addition, it has been reported that low molecular weight azide containing compounds may be unstable, and therefore, limitations on the commercial transportation of such compounds especially across international borders, may be difficult or prohibited unless certain stability criteria can be met. Therefore, when used as a feedstock, 4-azidobutylamine is reportedly produced on-site and used immediately before it begins to degrade. If the material cannot be used immediately, it reportedly must be stored at low temperatures (below 10° C.) under an inert atmosphere such as nitrogen. In addition, because of the potentially variable quality of even freshly prepared 4-azidobutylamine, manufacturing processes must often use a large molar excess of 4-azidobutylamine to ensure reaction completion.

A need exists for stabilized forms of 4-azidobutylamine that can be stored for longer periods of time under ambient conditions. A need also exists for forms of 4-azidobutylamine that have increased heat stability. A need also exists for stabilized forms of 4-azidobutylamine that have a decreased propensity for explosive degradation and can be commercially transported in bulk.

Described herein are various forms of 4-azidobutylamine that show increased chemical, heat, and storage stability.

In one illustrative embodiment of the invention, 4-azidobutylamine salts and derivatives are described herein. In another embodiment, processes for producing 4-azidobutylamine and salts thereof are described herein.

### BRIEF DESCRIPTION OF THE DRAWINGS

FIG. 1 shows the ¹H NMR spectra of 4-azidobutylamine after storing for 4 weeks; (A) 4-azidobutylamine prepared according to the processes described herein; (B) 4-azidobutylamine prepared according to conventional processes that contains residual DCM. The 4-azidobutylamine prepared according to the processes described herein was stored for an additional 6 weeks (10 weeks total) and did not show any differences by ¹H NMR.

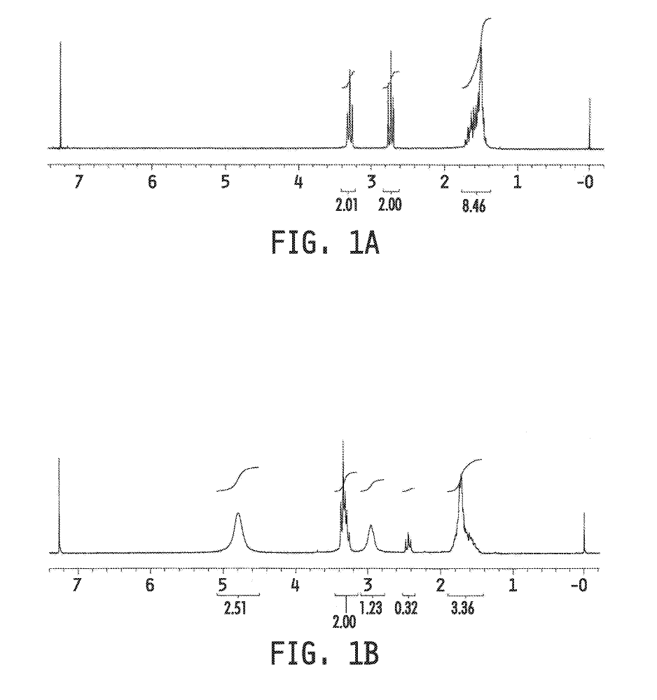

FIG. 2 shows the ¹³C NMR spectra of 4-azidobutylamine after storing for 4 weeks; (A) 4-azidobutylamine prepared according to the processes described herein; (B) 4-azidobutylamine prepared according to conventional processes that contains residual DCM. The 4-azidobutylamine prepared according to the processes described herein was stored for an additional 6 weeks (10 weeks total) and did not show any differences by ¹³C NMR.

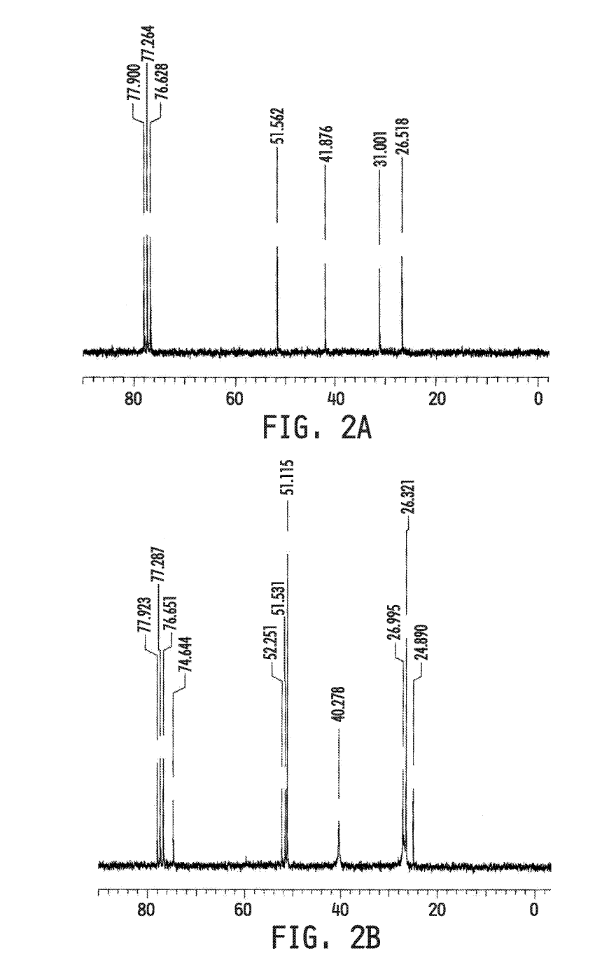

### DETAILED DESCRIPTION

In one illustrative embodiment of the invention, 4-azidobutylamine salts and derivatives are described herein.

It has been discovered herein that isolated salts of 4-azidobutylamine are more stable than conventional free-base forms of 4-azidobutylamine Salts of 4-azidobutylamine are observed to have improved storage characteristics. Salts of 4-azidobutylamine are also observed to have increased heat stability. The forced decomposition of salts of 4-azidobutylamine are also observed to release lower amounts of energy. Without being bound by theory, it is believed herein that the increased heat stability and the decreased energy release observed by the salts of 4-azidobutylamine described herein will translate into an improved profile for commercial transportation in bulk.

In another embodiment, salts of 4-azidobutylamine are described herein and are formed from one or more of nitrate, hydroiodide, hydrofluoride, chlorosulfonate, butyrate, maleate, propionate, pyruvate, lactate, hemioxalate, oxalate, hemitartrate, tartrate, hemisulfate, sulfate, formate, ⅓ citrate, ⅔ citrate, citrate, mesylate, hydrobromide, hemifumarate, fumarate, borate, hemimalonate, malonate, tosylate, benzoate, phosphate, and acetate, and combinations thereof.

In another embodiment, salts of 4-azidobutylamine are described herein and are formed from one or more of hemioxalate, oxalate, hemitartrate, tartrate, hemisulfate, sulfate, formate, ⅓ citrate, ⅔ citrate, citrate, mesylate, hydrobromide, hemifumarate, fumarate, borate, hemimalonate, malonate, tosylate, benzoate, phosphate, and acetate, and combinations thereof.

In another embodiment, salts of 4-azidobutylamine are described herein and are formed from one or more of hemioxalate, oxalate, hemitartrate, tartrate, hemisulfate, sulfate, formate, ⅓ citrate, ⅔ citrate, citrate, mesylate, hydrobromide, hemifumarate, fumarate, borate, hemimalonate, malonate, tosylate, benzoate, phosphate, and acetate, and combinations thereof.

In another embodiment, salts of 4-azidobutylamine are formed from one or more of tosylate, benzoate, phosphate, and acetate, and combinations thereof.

In another embodiment, salts of 4-azidobutylamine are formed from one or more of benzoate, phosphate, and acetate, and combinations thereof.

In another embodiment, salts of 4-azidobutylamine are formed from one or more of tosylate, benzoate, phosphate, and acetate, and combinations thereof.

In another embodiment, salts of 4-azidobutylamine are formed from one or more of benzoate, phosphate, and acetate, and combinations thereof.

In another embodiment, salts of 4-azidobutylamine are formed from phosphate.

In another embodiment, salts of 4-azidobutylamine are described herein that decompose with an energy of less than about 1000 J/g, less than about 900 J/g, or less than about 800 J/g.

In another embodiment, salts of 4-azidobutylamine are described herein that exhibit a exotherm, illustratively as determined by DSC, that starts at a temperature of about 100° C. or greater, about 110° C. or greater, about 120° C. or greater, about 125° C. or greater, about 130° C. or greater, about 140° C. or greater, about 150° C. or greater, about 160° C. or greater, about 170° C. or greater, about 175° C. or greater, about 180° C. or greater, or about 185° C. or greater.

In another embodiment, derivatives of 4-azidobutylamine are described herein, including but not limited to carbamates such as tert-butoxycarbonyl derivatives, benzyloxycarbonyl derivatives, and the like. In another embodiment, derivatives of 4-azidobutylamine are described herein, including but not limited to imides such as phthalimido derivatives, and the like.

It is appreciated herein that the salts and derivatives of 4-azidobutylamine that are solids may be advantageous and allow more ready isolation, handling, storage, and commercial transport than liquid salts and derivatives of 4-azidobutylamine.

In another illustrative embodiment, processes for producing 4-azidobutylamine and salts thereof are described herein.

Current processes for producing 4-azidobutylamine include chlorinated solvents, such as dichloromethane (DCM). It has been unexpectedly discovered herein that residual DCM in the 4-azidobutylamine free base destabilizes the 4-azidobutylamine and accelerates decomposition, leading to both poor quality and stability, short storage life, and commercial transportation limitations. It has also been unexpectedly discovered herein that a substantially chlorinated solvent-free manufacturing process and/or substantially chlorinated solvent-free isolation process provides 4-azidobutylamine with improved stability, and longer storage life. Substantially chlorinated solvent-free manufacturing processes and/or substantially chlorinated solvent-free isolation processes provide 4-azidobutylamine that is stable at room temperature, and that may be stored for long periods of time, including weeks, months or longer. It is appreciated herein that the stability of the 4-azidobutylamine can be further increased by storing under an inert atmosphere such as nitrogen gas, or at a lower temperature.

In another embodiment, 4-azidobutylamine free base is prepared in a chlorinated solvent-free process.

In another embodiment, 4-azidobutylamine free base is prepared in an organic solvent-free process.

In another embodiment, 4-azidobutylamine free base is isolated in a chlorinated solvent-free process.

In another embodiment, 4-azidobutylamine free base is isolated in an organic solvent-free process.

In another embodiment, 4-azidobutylamine free base is prepared in an organic solvent-free process. In conventional processes, the 4-azidobutylamine is isolated by extraction into an organic solvent, that organic solvent must be subsequently removed, such as by evaporation, distillation, and the like, often leading to lower yields through processing losses due to co-evaporation. Such removal of the organic solvent also often requires heating, which may be precluded on large manufacturing scales due to safety concerns arising from potential instability or rapid decomposition reported for low molecular weight azides. Evaporative techniques may be performed without heating, but may not lead to sufficient removal of residual solvent without substantially lowering the yield of the 4-azidobutylamine.

In another embodiment, 4-azidobutylamine free base is isolated in a chlorinated solvent-free process. Illustratively, the process comprises one or more of the following steps, and any combination thereof: (a) dissolving 4-azidobutylamine or a salt thereof into an acidic aqueous solution; (b) extracting the acidic aqueous solution with an organic solvent that is substantially free of or free of any non-chlorinated solvent; (c) raising the pH of the aqueous solution by adding a base; and/or (d) removing the formed layer of neat 4-azidobutylamine.

In another embodiment, 4-azidobutylamine free base is isolated in a chlorinated solvent-free process. Illustratively, the process comprises one or more of the following steps, and any combination thereof: (a) dissolving 4-azidobutylamine or a salt thereof into an acidic aqueous solution; (b) extracting the acidic aqueous solution with an organic solvent that is substantially free of or free of any non-chlorinated solvent; (c) raising the pH of the aqueous solution by adding a base; (d) extracting the basic aqueous solution with an organic solvent that is substantially free of or free of any non-chlorinated solvent; and/or (e) evaporating the organic solvent to isolate 4-azidobutylamine.

Additional embodiments of the invention are described by the following illustrative non-limiting clauses:

A process for preparing 4-azidobutylamine, comprising the step of isolating the 4-azidobutylamine from a solvent that is substantially free or free of a chlorinated solvent.

The process of the preceding clause wherein the solvent is N, N-dimethyformamide or MTBE, or a mixture thereof.

A process for preparing 4-azidobutylamine, comprising the step of isolating the 4-azidobutylamine from a mixture that is substantially free or free of a chlorinated solvent, or substantially free or free of an organic solvent.

The process of any one of the preceding clauses wherein the mixture comprises an aqueous solution and the 4-azidobutylamine is isolated therefrom without using an organic solvent.

The process of any one of the preceding clauses wherein the chlorinated solvent is dichloromethane.

A process for preparing 4-azidobutylamine, comprising the step of isolating the 4-azidobutylamine from an aqueous solution without any organic solvent.

The process of any one of the preceding clauses further comprising the step of raising the pH of the mixture.

The process of any one of the preceding clauses further comprising the step of separating an aqueous layer from the mixture.

A process for preparing 4-azidobutylamine, the process comprising

(a) adding a solution of sodium azide to a solution of 4-dibromobutane in N, N-dimethyformamide to form a mixture;

(b) heating the mixture at a temperature above ambient temperature, such as about 80 to about 90° C., for a predetermined time, such as about 12 h;

(c) adding an organic solvent that is substantially free of or free of chlorinated solvent, such as MTBE;

(d) isolating an organic layer;

(e) adding a solution of triphenylphosphine and an acid to the organic layer;

(f) heating the mixture at a temperature above ambient temperature, such as about 25 to about 35° C., for a predetermined time, such as about 12 h;

(g) adding sodium hydroxide to the mixture;

(h) isolating the organic layer containing 4-azidobutylamine; and

(i) degassing and drying the organic layer.

Isolated 4-azidobutylamine substantially free of or free of a chlorinated solvent.

Isolated 4-azidobutylamine prepared according to a process described herein.

An isolated salt of 4-azidobutylamine, where the salt comprises, consists essentially of, or consists of a nitrate, fluoride, bromide, iodide, sulfate, chlorosulfonate, methanesulfonate, toluenesulfonate, phosphate, phosphonate, oxalate, borate, citrate, malonate, formate, butyrate, maleate, propionate, pyruvate, benzoate, or lactate, or a combination thereof.

An isolated salt of 4-azidobutylamine, where the salt comprises, consists essentially of, or consists of a nitrate, fluoride, bromide, iodide, sulfate, chlorosulfonate, methanesulfonate, toluenesulfonate, phosphate, phosphonate, oxalate, borate, citrate, malonate, formate, butyrate, maleate, propionate, pyruvate, benzoate, or lactate, or a combination thereof.

A composition consisting essentially of an acid addition salt of 4-azidobutylamine, where the composition is substantially free of or free of a chlorinated solvent.

The composition of any one of the preceding clauses wherein the acid is selected from the group consisting of methanesulfonic acid, sulfuric acid, phosphoric acid, oxalic acid, toluenesulfonic acid, boric acid, and citric acid, and combinations thereof.

The composition of any one of the preceding clauses wherein the acid is selected from the group consisting of methanesulfonic acid, sulfuric acid, phosphoric acid, oxalic acid, toluenesulfonic acid, boric acid, and citric acid, and combinations thereof.

The composition of any one of the preceding clauses wherein the acid is selected from the group consisting of hydroiodide, hydrobromide, hydrofluoride, nitric acid, chlorosulfonic acid, malonic acid, formic acid, butyric acid, maleic acid, propionic acid, pyruvic acid, benzoic acid, and lactic acid, and combinations thereof.

A composition consisting essentially of an acyl derivative of 4-azidobutylamine.

The composition of the preceding clause wherein the acyl derivative is a Boc or phthalimido derivative.

The salt or composition of any one of the preceding clauses having an exotherm by DSC starting at greater than about 150° C., or greater than about 175° C., or greater than about 180° C.

The salt or composition of any one of the preceding clauses being capable of long-term storage, such as for more than about 10 days, more than about 20 days, more than about 30 days, more than about 45 days, more than about 60 days, or more than about 90 days at ambient temperature.

The salt or composition of any one of the preceding clauses wherein the ambient temperature is between about 15° C. and about 30° C., between about 15° C. and about 25° C.; or between about 15° C. and about 20° C.

The salt or composition of any one of the preceding clauses wherein after long-term storage, no more than 1%, no more than 2%, no more than 3%, no more than 4%, or no more than 5% decomposition is observed

A process for preparing 4-azidobutylamine, the process comprising

(a) adding a solution of sodium azide to a solution of 4-dibromobutane in N, N-dimethyformamide to form a mixture;

(b) heating the mixture at a temperature above ambient temperature, such as about 80 to about 90° C., for a predetermined time, such as about 12 h;

(c) adding a solution of triphenylphosphine and an acid;

(d) heating the mixture at a temperature above ambient temperature, such as about 25 to about 35° C., for a predetermined time, such as about 12 h;

(e) raising the pH of the mixture to separate the 4-azidobutylamine;

(f) isolating the 4-azidobutylamine; and

(g) adding solid sodium hydroxide to the 4-azidobutylamine.

A 4-azidobutylamine prepared according to any process described herein.

A 4-azidobutylamine salt prepared according to any process described herein.

A 4-azidobutylamine derivative prepared according to any process described herein.

A process for preparing a compound of the formula

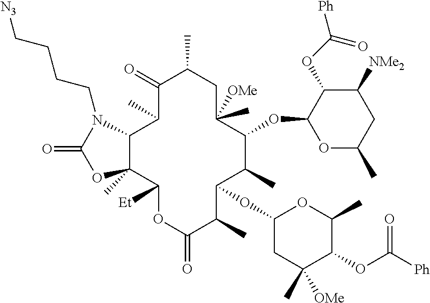 [SMILES: CC[C@H]1OC(=O)[C@H](C)[C@H](O[C@H]2C[C@@](C)(OC)[C@@H](OC(=O)c3ccccc3)[C@H](C)O2)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCN=[N+]=[N-])C(=O)O[C@]12C]

or a salt thereof; the process comprising adding a 4-azidobutylamine described herein, or optionally isolating 4-azidobutylamine from a 4-azidobutylamine salt described herein, and adding the isolated 4-azidobutylamine to a compound of the formula:

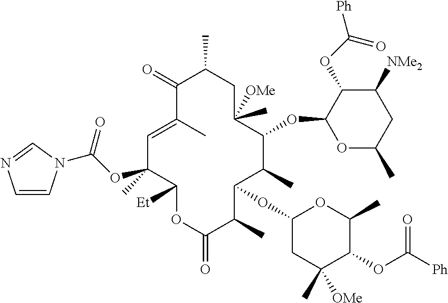 [SMILES: CC[C@H]1OC(=O)[C@H](C)[C@H](O[C@H]2C[C@@](C)(OC)[C@@H](OC(=O)c3ccccc3)[C@H](C)O2)[C@H](C)[C@@H](O[C@@H]2O[C@H](C)C[C@H](N(C)C)[C@H]2OC(=O)c2ccccc2)[C@@](C)(OC)C[C@@H](C)C(=O)/C(C)=C/[C@]1(C)OC(=O)n1ccnc1]

or a salt thereof.

A process for preparing solithromycin, the process comprising adding a 4-azidobutylamine described herein, or optionally isolating 4-azidobutylamine from a 4-azidobutylamine salt described herein, and adding the isolated 4-azidobutylamine to a compound of the formula:

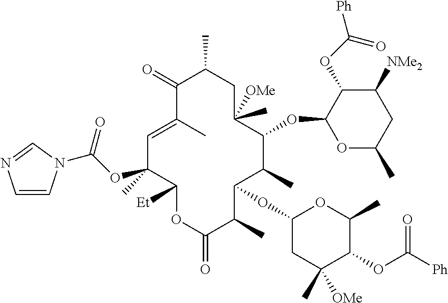 [SMILES: CC[C@H]1OC(=O)[C@H](C)[C@H](O[C@H]2C[C@@](C)(OC)[C@@H](OC(=O)c3ccccc3)[C@H](C)O2)[C@H](C)[C@@H](O[C@@H]2O[C@H](C)C[C@H](N(C)C)[C@H]2OC(=O)c2ccccc2)[C@@](C)(OC)C[C@@H](C)C(=O)/C(C)=C/[C@]1(C)OC(=O)n1ccnc1]

or a salt thereof, to prepare a compound of the formula

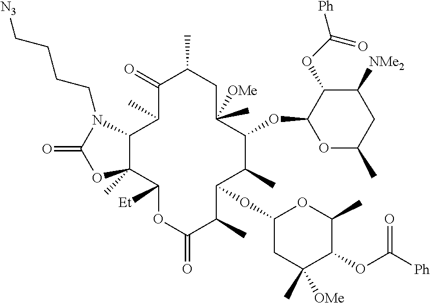 [SMILES: CC[C@H]1OC(=O)[C@H](C)[C@H](O[C@H]2C[C@@](C)(OC)[C@@H](OC(=O)c3ccccc3)[C@H](C)O2)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCN=[N+]=[N-])C(=O)O[C@]12C]

or a salt thereof.

The process of the preceding clause further comprising converting a compound of the formula:

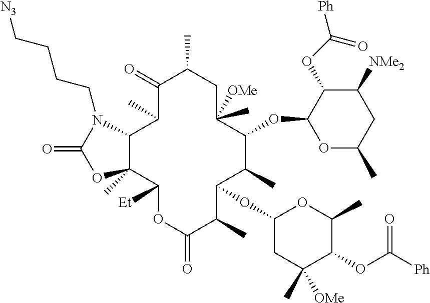 [SMILES: CC[C@H]1OC(=O)[C@H](C)[C@H](O[C@H]2C[C@@](C)(OC)[C@@H](OC(=O)c3ccccc3)[C@H](C)O2)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCN=[N+]=[N-])C(=O)O[C@]12C]

or a salt thereof, into a compound of the formula

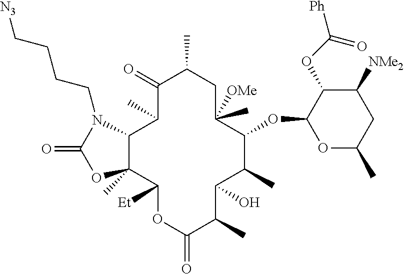 [SMILES: CC[C@H]1OC(=O)[C@H](C)[C@H](O)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCN=[N+]=[N-])C(=O)O[C@]12C]

or a salt thereof.

The process of any one of the preceding clauses further comprising converting a compound of the formula:

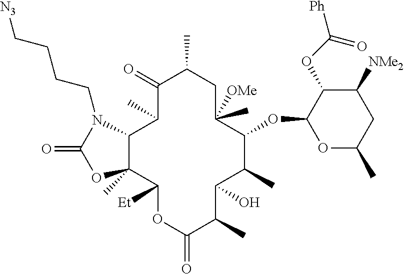 [SMILES: CC[C@H]1OC(=O)[C@H](C)[C@H](O)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCN=[N+]=[N-])C(=O)O[C@]12C]

or a salt thereof, into a compound of the formula

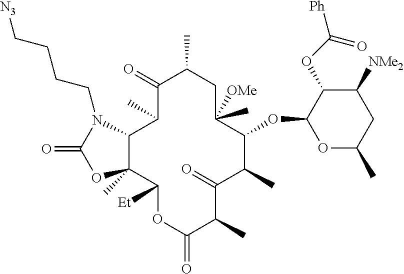 [SMILES: CC[C@H]1OC(=O)[C@H](C)C(=O)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCN=[N+]=[N-])C(=O)O[C@]12C]

or a salt thereof.

The process of any one of the preceding clauses further comprising converting a compound of the formula:

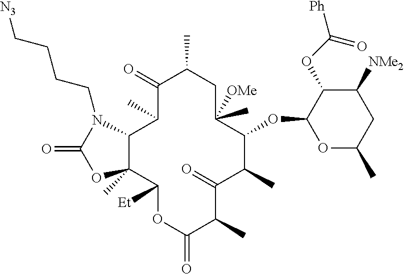 [SMILES: CC[C@H]1OC(=O)[C@H](C)C(=O)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCN=[N+]=[N-])C(=O)O[C@]12C]

or a salt thereof, into a compound of the formula

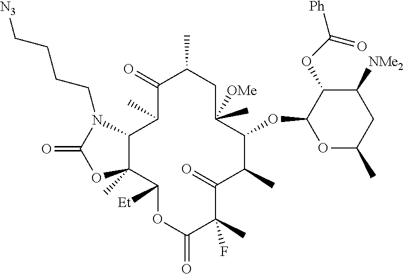 [SMILES: CC[C@H]1OC(=O)[C@@](C)(F)C(=O)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCN=[N+]=[N-])C(=O)O[C@]12C]

or a salt thereof.

The process of any one of the preceding clauses further comprising converting a compound of the formula:

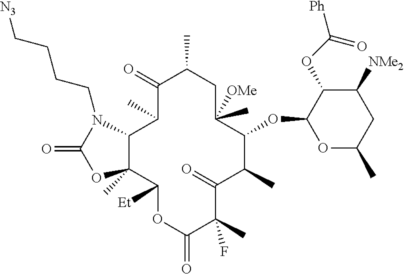 [SMILES: CC[C@H]1OC(=O)[C@@](C)(F)C(=O)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCN=[N+]=[N-])C(=O)O[C@]12C]

or a salt thereof, into a compound of the formula

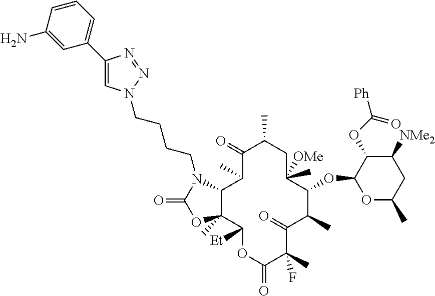 [SMILES: CC[C@H]1OC(=O)[C@@](C)(F)C(=O)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCn3cc(-c4cccc(N)c4)nn3)C(=O)O[C@]12C]

or a salt thereof.

The process of any one of the preceding clauses further comprising converting a compound of the formula:

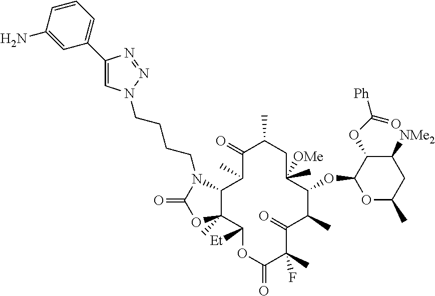 [SMILES: CC[C@H]1OC(=O)[C@@](C)(F)C(=O)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCn3cc(-c4cccc(N)c4)nn3)C(=O)O[C@]12C]

or a salt thereof, into solithromycin or a salt thereof.

A compound of the formula

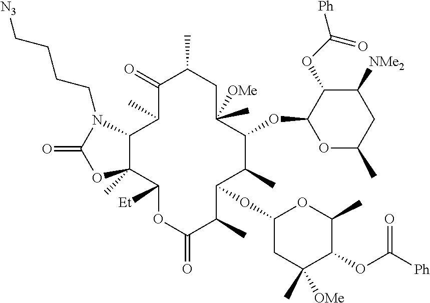 [SMILES: CC[C@H]1OC(=O)[C@H](C)[C@H](O[C@H]2C[C@@](C)(OC)[C@@H](OC(=O)c3ccccc3)[C@H](C)O2)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCN=[N+]=[N-])C(=O)O[C@]12C]

or a salt thereof, prepared according to the process of any one of the preceding clauses.

A compound of the formula

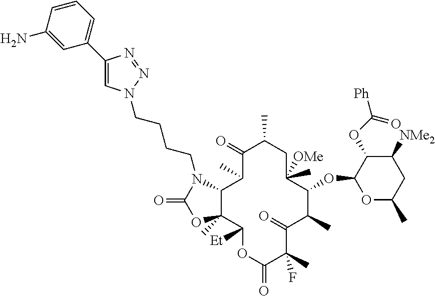 [SMILES: CC[C@H]1OC(=O)[C@@](C)(F)C(=O)[C@H](C)[C@@H](O[C@@H]2O[C@H](C)C[C@H](N(C)C)[C@H]2OC(=O)c2ccccc2)[C@@](C)(OC)C[C@@H](C)C(=O)[C@H](C)[C@H]2N(CCCCn3cc(-c4cccc(N)c4)nn3)C(=O)O[C@]12C]

or a salt thereof, prepared according to the process of any one of the preceding clauses.

Solithromycin or a salt thereof, prepared according to the process of any one of the preceding clauses.

Illustrative examples of processes used to produce the 4-azidobutylamine, and salts and derivatives thereof, including stabilized 4-azidobutylamine are described hereinbelow. It is to be understood that those examples are for illustrative purposes only, are not to be construed as limiting the invention in any way, and are not the sole embodiments of the invention nor the sole processes of making the described embodiments of the invention.

### EXAMPLES

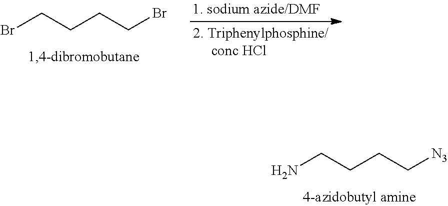 [SMILES: BrCCCCBr ; [N-]=[N+]=NCCCCN]

### Example

Substantially solvent free 4-azidobutylamine A sample of 100 g 1, 4-dibromobutane is dissolved in 300 mL N,N-dimethyformamide A sodium azide solution (125 g in 375 mL water) is added. The mixture is heated to 80-90° C. for 12 hrs. After completion of the reaction, the mixture is quenched with 1800 mL of water and 1200 mL of MTBE, resulting in the separation of the reaction products into layers. The organic layer containing the intermediate is removed. A concentrated HCl solution (120 mL in 600 mL water) and a triphenylphosphine (TPP) solution (200 g in 800 mL water) are added to the organic layer. The resulting mixture is stirred for 12 hours at 25-35° C. After completion of the reaction, solids are removed by filtration and the resulting mixture is separated into layers. The pH of the aqueous layer containing the product is raised with 300 g of sodium hydroxide. The final reaction mixture is filtered and separated. The product layer is degassed at 30-40° C., and then dried on sodium hydroxide to yield 35 g of 4-azidobutylamine (92.0-97.5% pure by gas chromatography, with a moisture content of 0.35-1.0%).

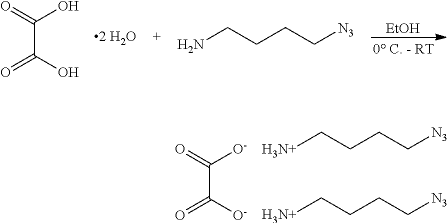 [SMILES: "EtOH" ; O=C(O)C(=O)O ; [N-]=[N+]=NCCCCN ; O=C([O-])C(=O)[O-] ; [N-]=[N+]=NCCCC[NH3+] ; [N-]=[N+]=NCCCC[NH3+]]

### Example

4-Azidobutylamine hemioxalate. To a solution clear of oxalic acid dihydrate (1.1 g, 8.76 mmol) in EtOH (10 mL) is slowly added 4-ABA (2.0 g, 17.52 mmol) in EtOH (2 mL) for a period of 30 min at 10° C. resulting in a white solid precipitate. The mixture is stirred at ambient temperature for 1 h, and diluted with Et₂O (40 mL). Stirring is continued for another 30 min. The precipitate is filtered, washed with Et₂O (10 mL) and dried under high vacuum to obtain 2.63 g (94%) of 4-ABA-oxalate salt as a white solid. ¹H NMR (200 MHz, D₂O): δ 3.25 (t, 2H, J=6.6 Hz), 2.89 (t, 2H, J=6.6 Hz), 1.68-1.43 (m, 4H); ¹³C NMR (50 MHz, D₂O): δ 171.95, 50.74, 39.29, 25.38, 24.42.

### Example

Salts of 4-azidobutylamine. Various other salts, including the salts described herein, are prepared as described herein, and using conventional processes.

### EXAMPLE

Derivatives of 4-azidobutylamine Various derivatives, including the derivatives described herein, are prepared as described herein, and using conventional processes.

### Example

Differential scanning calorimetry (DSC) may be performed using any conventional method. For example, DSC is performed using a Mettler-Toledo DSC-1 equipped with a FRSS Multi-thermocouple sensor and data acquisition. The sample is weighed in a 40 μL aluminum crucible with insert seating pin. The lid is punctured to insure no pressure build up and crimped to the crucible pan. The sample is inserted into the furnace well and seated in the sensor by way of the pin. The sample is equilibrated at 25° C. and heated to 250° C. at a rate of 5° C. per minute. Alternatively, DSC is performed on a Mettler-Toledo 822 DSC. The test sample is added to a gold plated, high pressure (sealed) test cell. An empty cell, is used as a reference pan, and is similarly prepared. The sample and reference pans are then placed into a furnace which is heated to 25° C. Once the pans have equilibrated with the furnace, the cells are heated at a constant rate (2-20° C./min) to 400° C. Microcomputer data logging is used to monitor the power output of the sample and the temperature in the oven.

The onset temperature is indicated by examining any upward deviation in the sample temperature from the reference temperature. The peak height or area under the curve indicates the magnitude of the energetic activity.

An endothermic event is a process in which heat is absorbed (negative heat flow) relative to the reference sample. Physical examples of endothermic events include, but are not limited to, melting, boiling, and solvent loss. Endothermic events are observed as a downward peak from the baseline.

An exothermic event is a process in which heat is given off (positive heat flow) relative to the reference sample. Physical examples of exothermic events include crystal formation and decomposition. Exothermic events are observed as an upward peak from the baseline.

A step change is a process where the baseline shifts. For glass transitions, the step change is usually endothermic and is consistent with a crystalline or ordered solid becoming amorphous.

The peak area of the endothermic event, exothermic event, and step change may be obtained by integration of the area bounded by a curve. The resulting transition is mathematically expressed as ΔH=KA, where ΔH is the enthalpy of transition and is equal to the product of K (a thermal constant) and A (area). The enthalpy of transition may be expressed Joules per gram, as calculated using conventional software, such as the STAR Software.

### Example

DSC of 4-Azidobutane-1-amine hemisulfate. 9.96 mg of sample was used. The sample was a glassy transparent solid. Most prominent features of the thermogram included a broad exotherm noted 140° C. that leads into an endothermic event at 157° C. A second endothermic event at 186° C. leads directly into a long exothermic decomposition. Thermogravimetric analysis or visual qualification of a melting point sample are used in determining if the events noted at ca. 140° C. are due to a decomposition pathway. Two more runs of the hemisulfate were performed to confirm results. Below are their thermograms. While some differences exist, all three thermograms show a noisy exotherm. Without being bound by theory, it is believed herein that the sample is hygroscopic. Accordingly, moisture content differences in the samples tested may account at least in part for the difference in each sample DSC. All three samples were characterized by black residue being exuded through the pin hole upon completion of the DSC run (decomposition).

### Example

DSC of 4-Azidobutane-1-amine phosphate. 3.51 mg of sample was used. The sample was a white opaque solid. Most prominent features of the thermogram included a sharp endotherm at 112° C., a slight but defined endothermic event at 123° C., followed by a broad endothermic event at 144° C. The three endothermic events were followed by a long exothermic decomposition. Visual qualification of a melting point sample is used in determining if the event noted at 112° C. is due to a melting of the sample. 4-Azidobutane-1-amine phosphate also passed the following standard UN Tests without decomposition: Friction Sensitivity Test, Drop hammer Test, Thermal Stability Test at 75° C., and Small Scale Burn Test.

### Example

4-Azidobutane-1-amine tosylate. 14.1 mg of sample was used. The sample was an opaque light brown solid. Most prominent features of the thermogram included a well-defined endotherm at 51° C. followed by a nondescript endotherm that began at 63° C. A very broad exothermic decomposition at 180° C. was noted. Thermogravimetric analysis or visual qualification of a melting point sample is used in determining the nature of the endothermic events. It is believed that the first endothermic event was a broad melting and the second endotherm represented an endothermic decomposition.

### Example

DSC of 4-Azidobutane-1-amine ⅓ citrate. 15.6 mg of sample was used. The sample was an opaque light colorless solid. Most prominent features of the thermogram included a broad two exotherms at 142° C. and at 193° C. Thermogravimetric analysis or visual qualification of a melting point sample are used in determining the nature of the exothermic events.

### Example

4-Azidobutane-1-amine mesylate. 6.71 mg of sample was used. The sample was an opaque light tan solid. Most prominent features of the thermogram included a broad endotherm with a sharp peak at 99° C. followed by a very broad endotherm that began at 223° C. Thermogravimetric analysis or visual qualification of a melting point sample are used in determining the nature of the endothermic events. It is believed that the first endothermic event is a broad melting and the second endotherm represents an endothermic decomposition.

### Example

DSC of 4-Azidobutane-1-amine hemioxalate. 5.12 mg of sample was used. The sample was a white solid. Most prominent features of the thermogram included a well-defined endotherm with a peak at 77° C. followed by a very broad endotherm that began at 190° C. and finally a large broad exotherm at 225° C. Thermogravimetric analysis or visual qualification of a melting point sample are used in determining the nature of the endothermic events. It is believed that the first endothermic event is a melting and the second endotherm and exotherm represent primary and secondary decompositions.

### Example

DSC of 4-Azidobutane-1-amine hemitartrate. 8.98 mg of sample was used. The sample was a clear, colorless oil. Most prominent features of the thermogram included a vague endotherm that started at ca. 180° C. that transitioned to a large broad exotherm at 235° C. Thermogravimetric analysis or visual qualification of a sample heated in a capillary tube was used in determining the nature of the endothermic and exothermic events.

### Example

DSC of 4-Azidobutane-1-amine borate. 5.25 mg of sample was used. The sample was an oily solid that become a waxy solid by scratching the glass vial containing the sample. Most prominent features of the thermogram included a vague sloping endotherm that started at ca. that transitioned to a large broad exotherm. Thermogravimetric analysis or visual qualification of a melting point sample was used in determining the nature of the endothermic and exothermic events.

### Example

DSC of 4-Azidobutane-1-amine acetate. 8.41 mg of sample was used. The sample was a light yellow oil. Most prominent features of the thermogram included a very broad endotherm that began at 152° C. and a broad exotherm at 224° C. Thermogravimetric analysis of a sample heated in a capillary tube was used in determining the nature of the endothermic events.

### Example

DSC of tert-butyl 4-Azidobutylcarbamate. 6.31 mg of sample was used. The sample was an oil. Most prominent features of the thermogram included a long, vague sloping endotherm that transitioned to an exotherm. Thermogravimetric analysis or visual qualification of a sample heated in a capillary tube was used in determining the nature of the endothermic and exothermic events.

TABLE 1

| | | Start of | Energy of | |
|---|---|---|---|---|
| | Physical | Exotherm | Decomposition | |
| Example | Form | (T° C.) | (J/g) | mp |
| 4-azidobutylamine | liquid | 134° C. | 2398 | — |
| (free base) | | | | |
| 4-azidobutylamine | white | 154° C. | 2002 | >150° C. |
| hemioxalate | powder | | | |
| 4-azidobutylamine | clear | nd | nd | — |
| hemitartrate | oil | | | |
| 4-azidobutylamine | pale | 139° C. | 1798 | nd |
| hemisulfate | yellow | | | |
| | solid | | | |
| 4-azidobutylamine | amber | 97° C. | ~1369 | — |
| formate | oil | | | |
| 4-azidobutylamine | pale | 155° C. | 1229 | nd |
| citrate (1:3) | yellow | | | |
| | solid | | | |
| 4-azidobutylamine | white | 135° C. | 1173 | ~85° C. |
| mesylate | solid | | | |
| 4-azidobutylamine | amber | 124° C. | 1189 | nd |
| hydrobromide | solid | | | |
| 4-azidobutylamine | white | 126° C. | 1139 | nd |
| hemifumarate | crystals | | | |
| 4-azidobutylamine | yellow | 148° C. | 1171 | nd |
| borate | solid | | | |
| 4-azidobutylamine | pale | 169° C. | 1076 | nd |
| hemimalonate | yellow | | | |
| | solid | | | |
| 4-azidobutylamine | pale | 148° C. | 951 | ~80° C. |
| tosylate | yellow | | | |
| | solid | | | |
| 4-azidobutylamine | pale | 131° C. | 722 | nd |
| benzoate | yellow | | | |
| | solid | | | |
| 4-azidobutylamine | white | 195° C. | 684 | 130-150° C. |
| phosphate (1:3) | crystals | | | |
| 4-azidobutylamine | oil | nd | nd | — |
| acetate | | | | |
| N-boc-4- | liquid | 123° C. | ~948 | — |
| azidobutylamine | | | | |
| N-phthalimido-4- | solid | nd | nd | nd |
| azidobutylamine | | | | |

nd = not determined.

### Example

Preparation of 2′,4″-di-O-benzoyl-11-N-(4-Azidobutyl)-6-O-methylerythromyc in A 11,12-cyclic carbamate. 10,11-anhydro-2′,4″-di-O-benzoyl-12-O-imidazolylcarbonyl-6-O-methylerythromycin A is prepared according to WO 2009/055557, the disclosure of which is incorporated herein by reference. DMF (50 mL) is added to 10,11-anhydro-2′,4″-di-O-benzoyl-12-O-imidazolylcarbonyl-6-O-methylerythromycin A (10 g) at 25° C. to 35° C.

Illustrative salts of 4-azidobutylamine that can be used to prepare compounds described herein include, but are not limited to, nitrate, hydroiodide, hydrofluoride, hydrochloride, chlorosulfonate, butyrate, maleate, propionate, pyruvate, lactate, hemioxalate, oxalate, hemitartrate, tartrate, hemisulfate, sulfate, formate, ⅓ citrate, ⅔ citrate, citrate, mesylate, hydrobromide, hemifumarate, fumarate, borate, hemimalonate, malonate, tosylate, trifluoroacetate, benzoate, phosphate, and acetate salts, and combinations thereof.

4-Azidobutylamine prepared according to any process described herein or any salt of 4-azidobutylamine described herein, and/or, or any combination of the foregoing, (4.4 g) and DBU (1.5 g) are added to the reaction mixture, and stirred at 25° C. to 35° C. until the reaction was complete. The mixture is then treated with cold water, and the resulting solid precipitate is collected. The solid is treated with dichloromethane followed by extraction and removal of solvent to give the title compound.

### Example

Preparation of 11-N-(4-Azidobutyl)-5-(2′-benzoyldesosaminyl)-3-hydroxy-6-O-methylerythronolide A 11,12-cyclic carbamate. Acetone (10 mL) is added to 2′,4″-di-O-benzoyl-11-N-(4-Azidobutyl)-6-O-methylerythromycin A 11,12-cyclic carbamate (5 g) to obtain a clear solution at 25° C. to 35° C. Dilute HCl (10 mL) is added to the reaction mixture and it was stirred for 24 hours at ambient temperature. After the completion of the reaction, the reaction mixture is extracted with ethyl acetate and treated with a sodium hydroxide solution to give the title compound.

### Example

Preparation of 11-N-(4-Azidobutyl)-5-(2′-benzoyldesosaminyl)-3-oxo-6-O-methylerythronolide A 11,12-cyclic carbamate. Oxidation of 11-N-(4-azidobutyl)-5-(2′-benzoyldesosaminyl)-3-hydroxy-6-O-methylerythronolide A 11,12-cyclic carbamate (100 g, 0.1225 moles) with Dess-Martin periodinane (170 g, 0.400 moles) is carried out in dichloromethane at 10-15° C. The reaction mixture is stirred at 20-25° C. for 2 hr, then quenched with 5% aqueous sodium hydroxide solution. The organic layer is washed with water and a saturated solution of sodium chloride. The solvent is removed by distillation of the organic layer and the product is isolated from a mixture of diisopropyl ether and hexane. The separated solid is filtered and dried under vacuum at 30-35° C. to give the title compound.

### Example

Preparation of 11-N-(4-Azidobutyl)-5-(2′-benzoyldesosaminyl)-3-oxo-2-fluoro-6-O-methylerythronolide A, 11,12-cyclic carbamate. To a solution of 11-N-(4-azidobutyl)-5-(2′-benzoyldesosaminyl)-3-oxo-6-O-methylerythronolide A 11,12-cyclic carbamate (5 g) in tetrahydrofuran (400 mL) is added 7.3 mL of potassium tert-butoxide followed by addition of 2 g of N-fluorobenzenesulfonimide After about 1 hour, the mixture is quenched with water followed by extraction with dichloromethane. The organic layers are separated and concentrated to obtain the title compound.

### Example

11-N-(3-amino-phenyl-1-yl-[1,2,3]-triazole-1-yl]butyl)-5-(2′-benzoyldesosaminyl)-3-oxo-2-fluoro-erythronolide A, 11,12-cyclic carbamate. 11-N-(4-azidobutyl)-5-(2′-benzoyldesosaminyl)-3-oxo-2-fluoro-6-O-methylerythronolide A, 11,12-cyclic carbamate (10 g), 3-ethynylphenylamine (2.11 g), copper iodide (0.3 g) and diisopropylethylamine (15.5 g) are added to acetonitrile (200 mL) and stirred for 20 hours at room temperature. After completion of the reaction, the reaction mixture is quenched with dilute HCl and extracted with dichloromethane. The organic layer is neutralized with a bicarbonate solution, dried and concentrated to obtain the title compound.

### Example

11-N-(3-amino-phenyl-1-yl-[1,2,3]-triazole-1-yl]butyl)-5-desosaminyl-3-oxo-2-fluoro-erythronolide A, 11,12-cyclic carbamate (solithromycin). 11-N-(3-amino-phenyl-1-yl-[1,2,3]-triazole-1-yl]butyl)-5-(2′-benzoyldesosaminyl)-3-oxo-2-fluoro-erythronolide A, 11,12-cyclic carbamate (6 g) is dissolved in methanol (60 mL) and heated at reflux for 7 hours. After the completion of reaction, the mixture is concentrated, diluted with diisopropylether (30 mL) and stirred at ambient temperature for 2 hours. The resulting solid is collected by filtration.

1. A process for preparing 4-azidobutylamine, the process comprising isolating the 4-azidobutylamine from a solvent that is substantially free or free of a chlorinated solvent.

2. The process of claim 1 wherein the solvent is N, N-dimethyformamide or MTBE, or a mixture thereof.

3. The process of claim 1 wherein the solvent is an aqueous solution.

4. The process of claim 3 further comprising raising the pH of the mixture.

5. The process of claim 4 further comprising separating an aqueous layer from the mixture.

6. (canceled)

7. Isolated 4-azidobutylamine substantially free of or free of a chlorinated solvent.

8. An isolated salt of 4-azidobutylamine, where the salt comprises a nitrate, fluoride, bromide, iodide, sulfate, chlorosulfonate, methanesulfonate, toluenesulfonate, phosphate, phosphonate, oxalate, borate, citrate, malonate, formate, butyrate, maleate, propionate, pyruvate, benzoate, or lactate, or a combination thereof.

9.-10. (canceled)

11. The salt of claim 8 comprising an acid, wherein the acid is selected from the group consisting of hydroiodide, hydrobromide, hydrofluoride, nitric acid, chlorosulfonic acid, malonic acid, formic acid, butyric acid, maleic acid, propionic acid, pyruvic acid, benzoic acid, and lactic acid, and combinations thereof.

12. The salt of claim 8 having an exotherm by DSC starting at about 150° C. or greater.

13. The salt of claim 8 being capable of long-term storage for about 10 days or more at ambient temperature.

14. The salt of claim 8 wherein after long-term storage, no more than 1% decomposition is observed.

15. (canceled)
